(19) United States
(12) Patent Application Publication
(10) Pub. No.: US 2008/0306248 A1
(43) Pub. Date: Dec. 11, 2008

(54) Conjugate of Biomacromolecule with Bioreductive and Preparative Method Thereof

(75) Inventors: Yiqiao Hu, Jiangsu (CN); Jinhui Wu, Jiangsu (CN); Lin Wu, Jiangsu (CN); Lingying Luo, Jiangsu (CN); Feng Zhi, Jiangsu (CN)

Correspondence Address: SCHMEISER, OLSEN & WATTS, 22 CENTURY HILL DRIVE, SUITE 302, LATHAM, NY, 12110, US

(73) Assignee: NANJING UNIVERSITY, Nanjing, Jiangsu 210093 (CN)

(21) Appl. No.: 11/916,539

(22) PCT Filed: Mar. 31, 2006
(86) PCT No.: PCT/CN2006/000582

§ 371 Date: Dec. 5, 2007

(30) Foreign Application Priority Data
Mar. 31, 2005 (CN) 200510038631.3
Mar. 31, 2006 (CN) PCT/CN06/000582

(51) Int. Cl. C07K 14/79

(52) U.S. Cl. 530/400; 530/402

(57) ABSTRACT

Conjugate of biomacromolecule with bioreductive which can be useful for treating tumor is provided. The biomacromolecule is selected from apo-transferrin, Fe-transferrin, Ru-transferrin, Ti-transferrin, Ga-transferrin, Pt-transferrin, somatostatin, EGF, folacin acid or transcobalamin, and the bioreductive agent is selected from quinones, aromatic nitrogen oxides, fatty nitrogen oxides, heterocyclic nitro compound, transition metal compound. Such conjugate can selectively target the tumor cells, and lower the toxicity of medicines and survivability of tumor cells, so that the conjugate can be used for delivery of anti-tumor compounds or treating tumors.

### FIELD OF THE INVENTION

This invention relates generally to the field of biopharmaceutical technology, more specifically to a conjugate of a biomacromolecule and a bioreductive, which is useful in cancer treatment.

### BACKGROUND OF THE INVENTION

Two of the most devastating problems in cancer treatment are drug-toxicity and drug-resistance. One way to solve the problem of drug-toxicity is to target drugs for delivery only to cancer cells. Many researcher are working to develop antibodies to delivery drugs to targeted cells, and this approach holds promise, but antibodies are not without problems. For example, antibodies often bind to normal tissues, and the also can damage blood vessels (e.g., vascular leak syndrome) and cause dangerous allergic reactions (e.g. anaphylaxis).

Transferrin is a kind of β1 globins with the molecular weight of about 77 kD, which accounts for 0.3%-0.5% of the plasma proteins. The main use of transferrin is delivering iron and transporting it into cells by the endocytosis mediated by the transferrin receptors on cell surfaces. And it also can transport other exogenous metal ions, such as Ru, Ti, Ga, Pt and so on. Transferrin was found 50 years ago, researchers recently have noticed that cancer cells need more irons for rapid growth than normal ones. Therefore, more transferrin receptors are expressed on their surfaces.

In hypoxia cells, bioreductive agent is activated by reductases and transformed into toxic products. This process occurs more easily in hypoxia cells and DT-diaphorases rich cells, which explains why bioreductive agents have high specificity for cancer cells.

Based on the characteristic of cancer cells mentioned above, this invention conjugates transferrin comprising Fe, Ru, Ti, Ga or Pt, somatostatin, EGF, folacin acid or transcobalamin with bioreductive agent, in order to delivery anti-tumor compounds for cancer treatment.

Research is also progressing in connection with the use of conjugates of transferrin and chemotherapy drugs, as described in U.S. Pat. Nos. 5,108,987; 5,000,935; 4,895,714 and 2,004,157,767. Conjugates of transferrin with doxorubicin, daunomycin, methotrexate, vincristin, 6-mercaptopurine, cytosinear abinoside, cyclophosphamide and radioactive iodine have high targeting and low toxicity.

### DETAILED DESCRIPTION OF THE INVENTION

The present invention relates to conjugates with a targeting agent and an antitumor agent for solving drug-toxicity and drug-resistance, more specifically with a biomacromolecule and a bioreductive agent.

The biomacromolecules according to the present invention include but are not limited to apo-transferrin, Fe-transferrin, Ru-transferrin, Ti-transferrin, Ga-transferrin, Pt-transferrin, somatostatin, EGF (epidermal growth factor), folacin acid and transcobalamin.

The bioreductive agents include but are not limited to:
1) Quinones: mitomycinC, diaziquon, streptonigrin, indoloquinone EO9 (3-hydroxy-aziridinyl-1-methyl-2-(1H-indole-4,7-indione)-propenol), RH1 (2,5-diaziridinyl-3-(hydroxymethyl)-6-methyl-1,4-benzoquinone), porfiromycin.

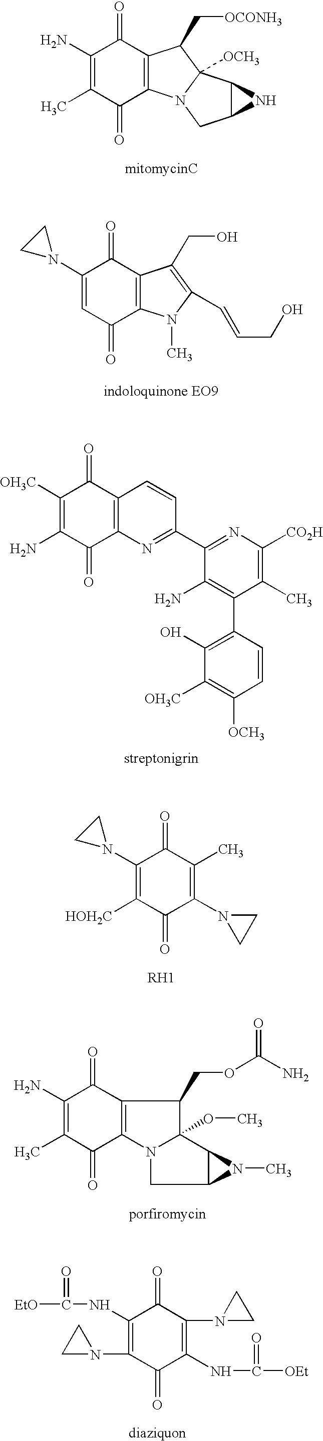

2) Aromatic N-oxides: Tirapazamine.

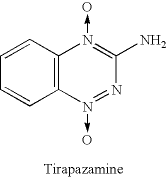

3) Aliphatic N-oxides:
   AQ4N (the di-N-oxide of 1,4-bis[{2-dimethylaminoethyl}-amino]5,8-dihydroxyanthracene-9,10-dione), Nitracrine N-Oxide.

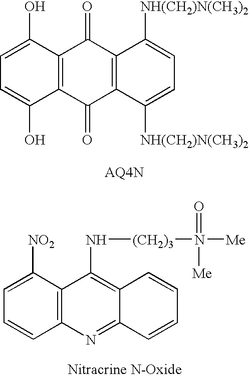

4) Nitroheterocyclic compounds:
   RSU1069 (1 [2-nitro-1-imidazolyl]-3-aziridinyl-2-ropanol), RB6145 (Beatrice S, Lloyd R. K. Methods Mol Med. 2004, 90, 515-42), CB1954(5-aziridin-1-yl-2,4-dinitrobenzamide), SN23862 (2,4-dinitrobenzam-ide mustard).

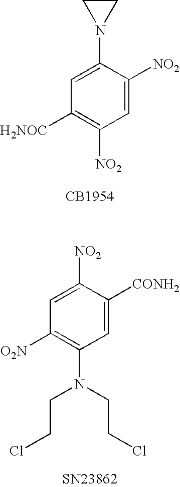

5) Transition metal complexes: SN24771.

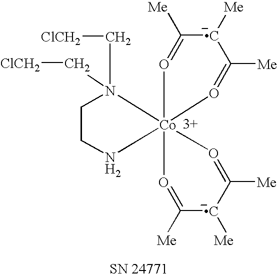

Preferably the biomacromolecules and the bioreductive agents are conjugated by a bond. Suitable bond include but are not limited to:
1. Covalent bond: glutaraldehyde, glutaric anhydride, disulfide coupling, thioester bond benzoyl hydrazone, N-hydroxy-succinimide, maleimide, etc.
II. Noncovalent bond: hydrogen bond, electrostatic interaction, coordination, etc.

This invention relates generally to a conjugate of biomacromolecule and bioreductive agent.

Whatever the bond is, the conjugate must keep bioactivity and can kill cancer cells without toxicity.

The significance of the conjugates with metal ions

a. Fe-Transferrin-Bioreductive Agents Conjugates
The peptide chain of serum transferrin folds into two globular lobes, which are similar in structure and respectively correspond to N-lobe and C-lobe. These two globular lobes are connected by a short peptide. Every lobe is divided into two domains with similar sizes, and these domains are constructed by alternate α-helixes and β-sheets. The N-lobe can be divided into N1 and N2 domains, which has disulfide bonds in but not between domains. C-lobe can be divided into C1 and C2 domains, which has no disulfide bond both in and between domains. With the existence of accompanying anions, some amino acid residues (Tyr, His, Asp) in the gap of two domains could form the binding side of Fe³⁺. Under physiological conditions, CO₃²⁻ is a kind of accompanying anions, and serum transferrin would form Fe-transferrin by reversible binding to 2Fe³⁺. This invention is to conjugate Fe-transferrin with bioreductive agent by covalent binding and noncovalent binding. Having double functions of targeting and anti-tumor, the conjugates can specifically kill the tumor hypoxia cells and inhibit tumor's growth and recurrence.

b. Ru-Transferrin-Bioreductive Agents Conjugates
Ru³⁺ has high anti-tumor effect, and is transported in blood by binding to transferrin. After injecting Ru-transferrin, Ru will be specifically absorbed by tumor cells. In addition, it has been found that Ru-transferrin has higher antitumor effect than Ru³⁺ in human colonic cancer. This invention is to conjugate Ru-transferrin with bioreductive agent by covalent binding and noncovalent binding. Having double functions of targeting and anti-tumor, the conjugates can specifically kill the tumor hypoxia cells and inhibit tumor's growth and recurrence.

c. Ti-Transferrin-Bioreductive Agents Conjugates
It is found that the binding between titanium and transferrin is similar to the binding between ion and transferrin. Ti-transferrin is also transported into tumor cells by transferrin receptor. And in the acid microenvironment of tumor cells, titanium will be released from transferrin and kill the tumor cells. This invention is to conjugate Ti-transferrin with bioreductive agent by covalent binding and noncovalent binding. Having double functions of targeting and anti-tumor, the conjugates can specifically kill the tumor hypoxia cells and inhibit tumor's growth and recurrence.

d. Ga-Transferrin-Bioreductive Agents Conjugates

Gallium is non-life element. After gallium is absorbed into the blood, it could quickly combine with serum transferrin to form a stable complex with the existence of accompanying anion —HCO³⁻. Similar to Fe-transferrin, Ga-transferrin has high affinity to transferrin receptors. This invention is to conjugate Ga-transferrin with bioreductive agent by covalent binding and noncovalent binding. On one hand the conjugate takes the bioreductive agents into the tumor cells to kill the tumor cells with the help of transferrin receptors, on the other hand the conjugate restrains the ingestion of iron of the tumor cells and increases the concentration of gallium in tumor cells, in the end reaches the object of targeting and better effect of drugs.

e. Pt-Transferrin-Bioreductive Agents Conjugates
Platinum can also bind to transferrin, and the binding site is the same to that of iron. Pt-transferrin will accumulate on the surface of tumor cells and be transported into tumor cells by the transferrin/transferrin receptors system. But the toxic effect of platinum to the hypoxia cells is not enough. Based on the synergy between bioreductive agents and anti-tumor drugs, this invention is to conjugate Pt-transferrin with bioreductive agent by covalent binding and noncovalent binding. The conjugates obviously enhance the targeting effects and decrease the drug dose.

Advantages of this invention compared to present techniques:

1) The transferrin-bioreductive conjugates have double function of targeting: a. target to transferrin receptors on the surface of cancer cells; b. target to hypoxic solid tumor. Thus, the purpose of decreasing drug-toxicity is achieved.

2) By the endocytosis of transferrin, anti-tumor agents are transported into cells, through which the drug-resistance of tumor cells was decreased and the therapeutic effect was increased.

3) The transferrin-bioreductive conjugates make a significant decrease in the dose required to achieve the same anti-tumor effect, and prevent the drug-resistance.

Thus, this invention is to prepare anti-tumor agents.

### EXAMPLE 1

### Fe-Transferrin-Mitomycin C conjugate

NaHCO₃ was added into 10 ml citric acid solution containing 20M apo-transferrin, adjust the pH to 7.4, stirred in ice water, dropwise 5 ml 10M NTA-FeCl₃, the mixture was stirred at 4° C., dialyzed against water, then freeze-dried. After that, the Fe-transferrin was prepared and the yield efficiency was 30%.

Glutaricanhydride (51.3 mg) was added to a stirred solution of MMC (50 mg) in dry tetrahydrofuran (40 mL), and the mixture was heated under nitrogen atmosphere at 50˜60° C. for 10˜20 hours. The solvent was evaporated, and the residue, after being dissolved in methanol (2 ml), was chromatographed on a Sephadex LH-20 column (2.5×97 cm) with methanol to give MMC having the 4-carbosybutyryl group attached at N-1α(90%). A solution of the carboxylic acid derivative of MNC thus obtained and N-hydroxysuccinimide (21.6 mg) was made in acetonitrite (3.4 ml). dicyclohexylcarbodiimide (155.6 mg) was added to this solution under cooling in an ice bath, and the mixture was stirred at 4° C. for 2 days. Ice water (7 ml) was added to the mixture, which was then filtered. The filtrate was diluted with water and extracted with chloroform. The extract was dried over sodium sulfated and subsequently evaporated. The residual material was treated with ethylacetate-n-hexane to give MMC-G-OSu (56%).

MMC-G-OSu (8.2 mg) in N,N-dimethylformamide (0.2 ml) was mixed with a solution of transferrin (100 mg) in 0.1M Na phosphate buffer (pH 7.0) (3 ml), and the mixture was allowed to stand at 4° C. over night. A very small amount of insoluble material was removed by centrifugation, and the supernatant was dialyzed at 4° C. to give Fe-Transferrin-mitomycin C conjugate (yeile efficiency was 20%).

The amount of MMC bound to transferrin was determined spectroscopically by measuring the absorbance at UV absorption maxima at 280 and 363 nm due to the chromophores of protein and MMC, respectively. When is mole ratio of MMC-G-OSu to transferrin is 43, the percent of MMC is 9.49%.

### EXAMPLE 2

### The Cytotoxicity of Fe-Transferrin-MMC Conjugate

Conjugate cytotoxicity was assessed using an MTT assay. Cells (SMMC-7721, L-02, etc.) were seeded at a density of 1×10⁴ cells/well 24 prior to the assay. At the start of the experiment the culture medium was removed and the conjugate (0-2 mg/lnl in complete medium) was added (100 ul). After 4 h, MTT (20 ul; 5 mg/ml in PBS) was added and the plates re-incubated for a further 5 h. the formazan crystals were dissolved in DMSO and the absorbance read at 550 nm using a microtitre plate reader. The results were expressed as viability(%) relative to a control containing no conjugate.

Results: the IC50 of conjugate and MMC to SMMC-7721 were 0.5 ug MMC/ml and 1.6 ug MMC/ml, respectively. Although the concentration of MMC or conjugate was up to 8 ugMMC/ml, the viability of L-02 was unchanged.

### EXAMPLE 3

### Transcellular Transport of Conjugate

The transcellular transport of conjugate was evaluated using Caco-2 cell monolayers. Caco-2 cells were maintained in plastic culture flasks. These stock cells were subcultivated before reaching confluence. The medium consisted of Dulbecco's Modified Eagle's Medium supplemented with 10% fetal bovine serum, 1% nonessential amino acid, 2 mM L-glutamine and 100 IU/mL penicillin-10 ug/mL streptomycin. The monolayer cultures were grown in an atmosphere of 5% CO2-95% O2 at 37° C. The cells were given fresh growth medium every 2 days. When the Caco-2 cells had reached confluence, they were harvested with 0.25 mM trypsin and 0.2% EDTA (0.5-1 min at 37° C.), resuspended, and seeded into a new flask, Caco-2 cells were used between passages 45 and 60. For the transport study, Caco-2 cells were seeded at a cell density of 8×10⁴ cells/cm² on 6-well (3-mm pores, 4.71-cm² growth area) Transwell™. The cell monolayers were fed a fresh growth medium every 2 days and were used at 16 to 21 days for the transport experiments. TEER was used to monitor the integrity of the monolayers. Monolayers with TEER above 350/cm² (after subtracting the back group value of the transwell) were used in the study.

Results: the transcellular transport of conjugate by caco-2 cells was 20% of total drug, while the transport freaction of MMC was only 5%. Compared with MMC, Fe-transferrin-MMC was more easily transcellular transported by caco-cell monolayers.

### EXAMPLE 4

### Tf-Fe-Diaziquon

Preparation procedure is parallel to example 1, and evaluation method is parallel to example 2 and 3.

### EXAMPLE 5

### Tf-Fe-Streptonigrin

Preparation procedure is parallel to example 1, and evaluation method is parallel to example 2 and 3.

### EXAMPLE 6

### Tf-Fe-EO9

Preparation procedure is parallel to example 1, and evaluation method is parallel to example 2 and 3.

### EXAMPLE 7

### Tf-Fe-RH1

Preparation procedure is parallel to example 1, and evaluation method is parallel to example 2 and 3.

### EXAMPLE 8

### Tf-Fe-Profiromycin

Preparation procedure is parallel to example 1, and evaluation method is parallel to example 2 and 3.

### EXAMPLE 9

### Tf-Fe-Tirapazamine

Preparation procedure is parallel to example 1, and evaluation method is parallel to example 2 and 3.

### EXAMPLE 10

### Tf-Fe-AQ4N

Preparation procedure is parallel to example 1, and evaluation method is parallel to example 2 and 3.

### EXAMPLE 11

### Tf-Fe-Nitracrine N-Oxidef

Preparation procedure is parallel to example 1, and evaluation method is parallel to example 2 and 3.

### EXAMPLE 12

### Tf-Fe-RSU1069

Preparation procedure is parallel to example 1, and evaluation method is parallel to example 2 and 3.

### EXAMPLE 13

### Tf-Fe-RB6145

Preparation procedure is parallel to example 1, and evaluation method is parallel to example 2 and 3.

### EXAMPLE 14

### Tf-Fe-CB1954

Preparation procedure is parallel to example 1, and evaluation method is parallel to example 2 and 3.

### EXAMPLE 15

### Tf-Fe-SN23862

Preparation procedure is parallel to example 1, and evaluation method is parallel to example 2 and 3.

### EXAMPLE 16

### Tf-Fe-SN24771

Preparation procedure is parallel to example 1, and evaluation method is parallel to example 2 and 3.

### EXAMPLE 17

### Tf-Ga-MMC

20 mg apo-transferrin was added into 9 ml 20 mM HAc solution containing 150 mM NaCl (pH 3.5), dripwise 3 mol Ga(NO₃)₃, then adjust pH to 7.4 by adding NaHCO₃, the mixture was dialyzed and freeze-dried. After that, the Ga-transferrin was prepared and the yield efficiency was 25%.

Other preparation procedure is parallel to example 1, and evaluation method is parallel to example 2 and 3.

### EXAMPLE 18

### Tf-Ga-MMC

Preparation procedure is parallel to example 17, and evaluation method is parallel to example 2 and 3, the yield efficiency was 40%.

### EXAMPLE 19

### Tf-Ga-Diaziquon

Preparation procedure is parallel to example 17, and evaluation method is parallel to example 2 and 3, the yield efficiency was 40%.

### EXAMPLE 20

### Tf-Ga-Streptonigrin

Preparation procedure is parallel to example 17, and evaluation method is parallel to example 2 and 3, the yield efficiency was 40%.

### EXAMPLE 21

### Tf-Ga-EO9

Preparation procedure is parallel to example 17, and evaluation method is parallel to example 2 and 3, the yield efficiency was 40%.

### EXAMPLE 22

### Tf-Ga-RH1

Preparation procedure is parallel to example 17, and evaluation method is parallel to example 2 and 3, the yield efficiency was 40%.

### EXAMPLE 23

### Tf-Ga-Profiromycin

Preparation procedure is parallel to example 17, and evaluation method is parallel to example 2 and 3, the yield efficiency was 40%.

### EXAMPLE 24

### Tf-Ga-Tirapazamine

Preparation procedure is parallel to example 17, and evaluation method is parallel to example 2 and 3, the yield efficiency was 40%.

### EXAMPLE 25

### Tf-Ga-AQ4N

Preparation procedure is parallel to example 17, and evaluation method is parallel to example 2 and 3, the yield efficiency was 40%.

### EXAMPLE 26

### Tf-Ga-Nitracrine N-Oxidef

Preparation procedure is parallel to example 17, and evaluation method is parallel to example 2 and 3, the yield efficiency was 40%.

### EXAMPLE 27

### Tf-Ga-RB6145

Preparation procedure is parallel to example 17, and evaluation method is parallel to example 2 and 3, the yield efficiency was 40%.

### EXAMPLE 28

### Tf-Ga-CB1954

Preparation procedure is parallel to example 17, and evaluation method is parallel to example 2 and 3, the yield efficiency was 40%.

### EXAMPLE 29

### Tf-Ga-SN23862

Preparation procedure is parallel to example 17, and evaluation method is parallel to example 2 and 3, the yield efficiency was 40%.

### EXAMPLE 30

### Tf-Ga-SN24771

Preparation procedure is parallel to example 17, and evaluation method is parallel to example 2 and 3, the yield efficiency was 40%.

### EXAMPLE 31

### Tf-Ti-MMC

Dissolve 20 mg apo-transferrin in 9 ml 20 mM acetic acid solution (pH=3.5) containing 150 mM NaCl. Add 3 mol nitrate titanium, and then add NaHCO₃ to adjust PH to 7.4. Dialyze and freeze dry, then get the Ti-transferrin. The yield rate is 30%.

Other preparation procedure is parallel to example 1, and evaluation method is parallel to example 2 and 3.

### EXAMPLE 32

### Tf-Ti-Diaziquone

Preparation procedure is parallel to example 31, and evaluation method is parallel to example 2 and 3

### EXAMPLE 33

### Tf-Ti-Rufocromomycin

Preparation procedure is parallel to example 31, and evaluation method is parallel to example 2 and 3

### EXAMPLE 34

### Tf-Ti-EO9

Preparation procedure is parallel to example 31, and evaluation method is parallel to example 2 and 3

### EXAMPLE 35

### Tf-Ti-RH1

Preparation procedure is parallel to example 31, and evaluation method is parallel to example 2 and 3

### EXAMPLE 36

### Tf-Ti-Porfiromycin

Preparation procedure is parallel to example 31, and evaluation method is parallel to example 2 and 3

### EXAMPLE 37

### Tf-Ti-Tirapazamine

Preparation procedure is parallel to example 31, and evaluation method is parallel to example 2 and 3

### EXAMPLE 38

### Tf-Ti-AQ4N

Preparation procedure is parallel to example 31, and evaluation method is parallel to example 2 and 3

### EXAMPLE 39

### Tf-Ti-Nitracrine N-Oxidef

Preparation procedure is parallel to sample 31, and evaluation method is parallel to sample 2 and 3.

### EXAMPLE 40

### Tf-Ti-RSU1069

Preparation procedure is parallel to example 31, and evaluation method is parallel to example 2 and 3

### EXAMPLE 41

### Tf-Ti-RB6145

Preparation procedure is parallel to example 31, and evaluation method is parallel to example 2 and 3

### EXAMPLE 42

### Tf-Ti-CB1954

Preparation procedure is parallel to example 31, and evaluation method is parallel to example 2 and 3

### EXAMPLE 43

### Tf-Ti-SN23862

Preparation procedure is parallel to example 31, and evaluation method is parallel to example 2 and 3

### EXAMPLE 44

### Tf-Ti-SN24771

Preparation procedure is parallel to example 31, and evaluation method is parallel to example 2 and 3.

### EXAMPLE 45

### Tf-Pt-MMC

Add NaHCO₃ into 10 ml citric acid solution containing 20M apotransferrin, and adjust pH to 7.4. Then the mixture was stirred in ice water. Add 5 ml 20M Cis-Diaminodichloroplatin Platinol and continue to stirred in ice water. Dialyze and freeze dry. After that, the Pt-transferrin was prepared and the yield efficiency was 20%.

Other preparation procedure is parallel to example 1, and evaluation method is parallel to example 2 and 3.

### EXAMPLE 46

### Tf-Pt-Diaziquone

Preparation procedure is parallel to example 45, and evaluation method is parallel to example 2 and 3.

### EXAMPLE 47

### Tf-Pt-Rufocromomycin

Preparation procedure is parallel to example 45, and evaluation method is parallel to example 2 and 3.

### EXAMPLE 48

### Tf-Pt-EO9

Preparation procedure is parallel to example 45, and evaluation method is parallel to example 2 and 3.

### EXAMPLE 49

### Tf-Pt-RH1

Preparation procedure is parallel to example 45, and evaluation method is parallel to example 2 and 3.

### EXAMPLE 50

### Tf-Pt-Porfiromycin

Preparation procedure is parallel to example 45, and evaluation method is parallel to example 2 and 3.

### EXAMPLE 51

### Tf-Pt-Tirapazamine

Preparation procedure is parallel to example 45, and evaluation method is parallel to example 2 and 3.

### EXAMPLE 52

### Tf-Pt-AQ4N

Preparation procedure is parallel to example 45, and evaluation method is parallel to example 2 and 3.

### EXAMPLE 53

### Tf-Pt-Nitracrine N-Oxidef

Preparation procedure is parallel to example 45, and evaluation method is parallel to example 2 and 3.

### EXAMPLE 54

### Tf-Pt-RSU1069

Preparation procedure is parallel to example 45, and evaluation method is parallel to example 2 and 3.

### EXAMPLE 55

### Tf-Pt-RB6145

Preparation procedure is parallel to example 45, and evaluation method is parallel to example 2 and 3.

### EXAMPLE 56

### Tf-Pt-CB1954

Preparation procedure is parallel to example 45, and evaluation method is parallel to example 2 and 3.

### EXAMPLE 57

### Tf-Pt-SN23862

Preparation procedure is parallel to example 45, and evaluation method is parallel to example 2 and 3.

### EXAMPLE 58

### Tf-Pt-SN24771

Preparation procedure is parallel to example 45, and evaluation method is parallel to example 2 and 3.

### EXAMPLE 60

### Tf-Ru-MMC

Add NaHCO₃ into 10 ml citric acid solution containing 20M apotransferrin. Then the mixture was stirred in ice water. Add 5 ml 20M ruthenium trichloride and continue to stirred in ice water. Dialyze and freeze dry. After that, the Ru-transferrin was prepared and the yield efficiency was 30%.

Other preparation procedure is parallel to example 1, and evaluation method is parallel to example 2 and 3.

### EXAMPLE 61

### Tf-Ru-Diaziquone

Preparation procedure is parallel to example 60, and evaluation method is parallel to example 2 and 3.

### EXAMPLE 62

### Tf-Ru-Rufocromomycin

Preparation procedure is parallel to example 60, and evaluation method is parallel to example 2 and 3.

### EXAMPLE 63

### Tf-Ru-EO9

Preparation procedure is parallel to example 60, and evaluation method is parallel to example 2 and 3.

### EXAMPLE 64

### Tf-Ru-RH1

Preparation procedure is parallel to example 60, and evaluation method is parallel to example 2 and 3.

### EXAMPLE 65

### Tf-Ru-Porfiromycin

Preparation procedure is parallel to example 60, and evaluation method is parallel to example 2 and 3.

### EXAMPLE 66

### Tf-Ru-Tirapazamine

Preparation procedure is parallel to example 60, and evaluation method is parallel to example 2 and 3.

### EXAMPLE 67

### Tf-Ru-AQ4N

Preparation procedure is parallel to example 60, and evaluation method is parallel to example 2 and 3.

### EXAMPLE 68

### Tf-Ru-Nitracrine N-Oxidef

Preparation procedure is parallel to example 60, and evaluation method is parallel to example 2 and 3.

### EXAMPLE 69

### Tf-Ru-RSU1069

Preparation procedure is parallel to example 60, and evaluation method is parallel to example 2 and 3.

### EXAMPLE 70

### Tf-Ru-RB6160

Preparation procedure is parallel to example 60, and evaluation method is parallel to example 2 and 3.

### EXAMPLE 71

### Tf-Ru-CB1954

Preparation procedure is parallel to example 60, and evaluation method is parallel to example 2 and 3.

### EXAMPLE 72

### Tf-Ru-SN23862

Preparation procedure is parallel to example 60, and evaluation method is parallel to example 2 and 3.

### EXAMPLE 73

### Tf-Ru-SN24771

Preparation procedure is parallel to example 60, and evaluation method is parallel to example 2 and 3.

### EXAMPLE 75

### Epidermal Growth Factor-Tirapazamine Conjugate

Dissolve 50 mg Epidermal growth factor in sodium phosphate buffered solution (pH=7.5) which contains 5 ml 0.1M NaCl. Then add 21.6 mg N-Hydroxysuccinimide (HOSu) and 155.6 mg DDC (Dicyclohexylcarbodiimide), and stir the mixture for 16 hours at 4° C. Then the mixture is dialyzed at 4° C. Add 30 mg tirapazamine, and stir for another 20 hours at 4° C. Then the mixture is dialyzed again at 4° C., freeze-dried. After that, the Epidermal growth factor-tirapazamine conjugate is prepared. The yield rate is 10%.

### EXAMPLE 76

### Flolic Acid-Porfiromycin Conjugate

Dissolve 50 mg flolic acid in sodium phosphate buffered solution (pH=7.5) which contains 5 ml 0.1M NaCl. Then add 21.6 mg N-Hydroxysuccinimide (HOSu) and 155.6 mg DDC (Dicyclohexylcarbodiimide), and stir the mixture for 16 hours at 4° C. Then the mixture is dialyzed at 4° C. Add 30 mg porfiromycin, and stir for another 20 hours at 4° C. Then the mixture is dialyzed again at 4° C., freeze-dried. After that, Flolic acid-Porfiromycin conjugate is prepared. The yield rate is 10%.

### EXAMPLE 77

### Transcobalamin-Tirapazamine Conjugate

Dissolve 50 mg transcobalamin in sodium phosphate buffered solution (pH=7.5) which contains 5 ml 0.1M NaCl. Then add 21.6 mg N-Hydroxysuccinimide (HOSu) and 155.6 mg DDC (Dicyclohexylcarbodiimide), and stir the mixture for 16 hours at 4° C. Then the mixture is dialyzed at 4° C. Add 30 mg tirapazamine, and stir for another 20 hours at 4° C. Then the mixture is dialyzed again at 4° C., freeze-dried. After that, transcobalamin-tirapazamine conjugate is prepared. The yield rate is 10%.

1. A conjugate comprising a biomacromolecule and a bioreductive agent, wherein said biomacromolecule is selected from the group consisting of apo-transferrin, Fe-transferrin, Ru-transferrin, Ti-transferrin, Ga-transferrin, Pt-transferrin, somatostatin, epidermal growth factor, folic acid and transcobalamin, and said bioreductive agent is selected from the group consisting of quinone, aromatic N-oxide, aliphatic N-oxide, nitroheterocyclic compound and transition-metal complexe, and wherein said homogeneous conjugates is substantially free of dimers, trimers and aggregates.

2. The conjugate according to claim 1, wherein said conjugate is used in treatment of cancer.

3. The conjugate according to claim 1, wherein said biomacromolecule is Fe-transferrin.

4. The conjugate according to claim 1, wherein said biomacromolecule is Ru-transferrin.

5. The conjugate according to claim 1, wherein said biomacromolecule is Ti-transferrin.

6. The conjugate according to claim 1, wherein said biomacromolecule is Ga-transferrin.

7. The conjugate according to claim 1, wherein said biomacromolecule is Pt-transferrin.

8. The conjugate according to claim 1, wherein said quinone is selected from the group consisting of diaziquon, streptonigrin, EO9, RH1 and porfiromycin.

9. The conjugate according to claim 1, wherein said aromatic N-oxide is tirapazamine.

10. The conjugate according to claim 1, wherein said aliphatic N-oxide is selected from the group consisting of AQ4N and Nitracrine N-Oxide.

11. The conjugate according to claim 1, wherein said nitroheterocyclic compound is selected from the group consisting of RSU1069, RB6145, CB1954 and SN23862.

12. The conjugate according to claim 1, wherein said transition-metal complex is SN24771.

13. The conjugate according to claim 1, wherein said biomacromolecule is Pt-transferrin, bioreductive agent is tirapazamine.

14. A method for making a conjugate comprising covalent binding and noncovalent binding, wherein said noncovalent binding is select from the group consisting of hydrogen bond, electrostaitic interaction and coordination.

15. The method according to claim 14, wherein said covalent binding is selected form the group consisting of glutaraldehyde, glutaric anhydride, disulfide coupling, thioester binding, benzoylhydrazone, N-hydroxy succinimide and maleimide.
